(19) United States
(12) Patent Application Publication
(10) Pub. No.: US 2026/0289005 A1
(43) Pub. Date: Sep. 24, 2026

(54) METHOD AND SYSTEM FOR PERSONAL HEALTH DATA MANAGEMENT

(71) Applicant: CUSHLA HEALTH SYSTEMS LTD, Dublin (IE)

(72) Inventors: Richard EGAN, Dublin (IE); Garrett HUSSEY

(21) Appl. No.: 19/477,474

(22) PCT Filed: Apr. 26, 2024
(86) PCT No.: PCT/IB2024/054087

§ 371 Date: Oct. 21, 2025

(30) Foreign Application Priority Data
Apr. 26, 2023 (GB) 2306103.9

(51) Int. Cl. G06F 21/62; G16H 10/60

(52) U.S. Cl. CPC G06F 21/6245; G16H 10/60

(57) ABSTRACT

A health information management system and method are disclosed. The system and method are data format agnostic and are directed to secure patient-controlled health information management. A patient-centric, interoperability platform is disclosed whereby fluid and continuous integration of universal validated healthcare information, which brokers all data securely, across all healthcare networks, including professional and patient initiated / ‘alternative’ interventions.

### FIELD OF THE INVENTION

The present invention relates to the field of health information management. In particular it relates to a data format agnostic system and method for secure patient-controlled health information management.

### BACKGROUND

The digitisation of healthcare records has seen benefits for practitioners, facilities and patients: for example, appointment booking systems, ‘self-serve’ diagnostic information, virtual consultation, document storage, progress monitoring and general facilities such as ‘speech to text’.

Some healthcare providers maintain ‘on premise’ management of health records. Others manage and deploy health services using infrastructure, platforms or software services (commonly referred to as IaaS, PaaS and SaaS respectively) hosted by third party providers over the internet and Cloud services (which include servers, storage, databases, networking, software, analytics and intelligence).

In practice, practitioner and facility patient management systems are generally specific to a practitioner or facility type. Such systems benefit from enhanced privacy, however the lack of access by and integration with other practices, and the restriction to specific aspects of healthcare, causes waste and inefficiencies on both an individual patient and on a global level: while a record of a visit to a health care professional, including GP, optician, physical therapist, and related information (such as test results and x-rays or MRI scans) is kept by the provider of that element of the treatment, full details of each interaction are generally not available to others outside the management system. Meanwhile patients have increasing opportunity to instigate ‘alternative’ therapies and health activities.

Integrated networks do exist in national health environments, although these generally do not include complimentary and supplementary healthcare interactions, especially impromptu patient-initiated therapies and ‘alternative’ therapies, although such therapies and interventions may be important in the health, diagnosis and determination of treatment of a patient.

Digitisation has also entailed increased administrative burden on practitioners, many of whom now undertake administrative tasks directly: for example, practitioners may take notes, triage questions, write prescriptions, organise referrals and process payments in addition to conducting consultations, diagnosis and patient care.

Where the patient is responsible for navigating the treatment pathway, and providing the requisite information from one provider to the next, information can be lost and errors introduced.

The Fast Healthcare Interoperability Resources (FHIR) standard defines how healthcare information is exchanged between different computer systems regardless of how it is stored in the respective systems. Building on previous data format standards from Health Level Seven (HLH7), incorporating web-based suite of API technology, (including a HTTP-based RESTful protocol, and a choice of JSON, XML or RDF for data representation), FHIR comprises resources (data formats and elements) and an application programming interface (API) for exchanging electronic health records (EHR) and facilitates interoperability between legacy health care systems, to make it easy to provide health care information to health care providers and individuals on a wide variety of devices from computers to tablets to cell phones, and to allow third-party application developers to provide medical applications which can be easily integrated into existing systems.

Integral to FHIR is that the root of the object graph is the facility. FHIR is organized by resource (e.g. patient, observation) which can be specified via defined FHIR profiles (for example, binding to a specific terminology). A collection of profiles can be published as an implementation guide (IG), such as the U.S. Core Data for Interoperability (USCDI). As FHIR is implemented on top of the HTTPS (HTTP Secure) protocol, FHIR resources can be retrieved and parsed by analytics platforms for real-time data gathering. Thus, healthcare organizations can gather real-time data from specified resource models and FHIR resources can be streamed to a data store where they can be correlated with other informatics data. Open-source implementations of FHIR data structures, servers, clients and tools include reference implementations from HL7 in a variety of languages, SMART on FHIR and HAPI-FHIR in Java. Other initiatives have built on FHIR to help medical research studies ask for (and if approved by the patient, receive) patient-level electronic health record data.

The evolution of patient rights has resulted in legislation relating to accessibility of patient data. For example introduction of the US 21^{st} Century Cures Act and related Centers for Medicare and Medicaid Services (CMS) Interoperability and Patient Access final rule, (CMS-9115-F) requires the use of FHIR by a variety of CMS-regulated payers (i.e. Medicare Advantage organizations, state Medicaid programs, and qualified health plans in the Federally Facilitated Marketplace). Specifically, the rule requires FHIR APIs to enable patient access, provider directories and payer-to-payer exchanges.

Furthermore, post-Covid pandemic initiatives to address inequalities in global health care have prioritized resilience and digitization of healthcare. There are further initiatives toward proactive health management that extend beyond reactive episodic care, including population segmentation and stratification, chronic disease care coordination plan, long-term health management plans, and consumer education.

Initiatives must however be coordinated, effective, consistent with evidence based clinical and data recommendations, reinforce national policy, be interoperable and reflect national digital maturity and healthcare capabilities, and protect individuals' privacy, security and safety.

Health information management systems and related interfaces have thus been disclosed with configuration for data sharing,

Many such systems refer to patient-centric functionality and entail means for aggregating electronic medical records and data and transmitting electronic medical records and data from electronic devices to a recipient electronic device.

In some systems the data source devices and user devices are connected to the health data server by a secure network. Such devices consist of any combination of wireless and wireline links (smart phone data-source., computer or laptop data-source, a server data-source, a tablet computer, a gaming device, a smart-television, a headset computer, a smartwatch, a body monitor device etcetera and any suitable number of any such data source devices).

Many systems claim means for; collating profile data; collating care plan data; integrating social networking data such as photographs with healthcare data; and integrating patient-generated fitness data.

Some systems receive, collate, store and display data relating to health screening, for example, data from blood glucose monitors, blood pressure monitors, weight scales, infusion pumps, temperature monitors, stethoscopes, blood coagulation meters, pulse oximeter monitors, apnea monitors, electrocardiogram monitors, fetal monitors and other biometrics.

Many such systems effect status alerts, assess a priority of patient need and assess a treatment outcome probability.

Some systems indicate the location of patient and/or the location of one or more healthcare provider; compare healthcare providers' suitability and/or quality via a score.

Systems are also known where the recipient electronic device belongs to a healthcare provider, a stakeholder, a social network, a peer-to-peer network, or a block chain system.

Generally, users have an identifier and each of the plurality of data is encrypted. The authentication information is one of a digital signature, a hash value, a digital certificate, a digital watermark, or an electronic postmark.

In some systems a fraudulent event is indicated.

In some systems authentication comprises broadcasting to a block chain system, transaction data corresponding, wherein the block chain system receives the transaction data, validates the transaction data, and adds a block to a chain indicating the transmission of the at least one of the alert, the electronic medical record of the patient, or the recommendation.

Some systems can be used along with other systems to implement a suite of technology-enabled data-driven solutions designed to augment and accelerate effective disease management and care. For example, a health data system can be integrated into local healthcare infrastructure to create a care management framework for delivering patient-centric and value-based care in a community and might be scaled a broader set of communities.

Some systems provide means to contextualized health profile whereby longitudinal physiological trends are coupled with behavioral, social and environmental data.

Some systems claim means for a variety of potential stakeholders, their associated data sources and approval for data sharing: for example, health data systems including health insurers/payers, pharmaceutical and medical device companies and research organisations as well as healthcare providers. Each stakeholder data source can choose the specific fields and elements, or subsets of data, which they approve to share, and the system can manage the approvals of identified data and/or identifiable data.

Some systems incorporate business rules configured in the respective agent modules and include naming conventions, data lineage tracking, permissible and non-permissible fields based on the ability to share identifiable data, sharing restrictions, and other stakeholder organization-specific rules for an agent to follow when managing data. In some systems, agent modules can encrypt data that will be transmitted to a server prior to leaving an environment and/or firewall of data source. In some systems, data that is not in accordance with the sharing business rules defined by the data source will remain in the environment of the data source and are not brought into the health data system by the agent modules. In some embodiments, this can include non-consented identifiable data, Thus, while systems have been proposed for obtaining, storing, curating and analysing health data and for providing patient access to health data, such systems are abstract and disparate. There is currently no effective technical means for fluid and continuous integration of universal healthcare information that brokers all data securely, across all healthcare networks, including professional and patient initiated/‘alternative’ interventions and validates source and transaction.

In addition, all such systems are healthcare facility-centric. None provide for a patient-centric operability platform.

The present invention provides such a platform. It comprises a patient-centric, interoperability platform whereby fluid and continuous integration of universal validated healthcare information, which brokers all data securely, across all healthcare networks, including professional and patient initiated/‘alternative’ interventions.

### SUMMARY OF THE INVENTION

According to a first aspect of the invention there is a method for an individual to manage universal health information.

Herein the term ‘universal health information’ may be construed to mean data/medical information covering some or all facets of one or more patient's health care.

According to an aspect the process comprises the steps of: creating a profile for an individual (referred to here as ‘patient’); authenticating an identity of the patient; providing an authorised user (the term ‘authorised user’ may be construed to mean any third party authorized by the patient) secure access to at least a portion of the patient's existing medical data file; collating user defined fields of medical data as agnostic data files; determining compatibility of the agnostic data files with at least one healthcare resource; and when compatibility has been authenticated, transmitting the agnostic data files to the present Interoperability platform by which the authorised user authorizes sharing the agnostic data securely via a shared database.

According to an aspect of the invention there is a data format agnostic system for secure individual patient controlled health information management, comprising devices arranged to: create a profile unique to each individual patient, authenticate the identity of the individual patient, provide at least one authorised user secure access to at least a portion of an existing medical data file of the individual patient, collate authorised user defined fields of medical data as agnostic data files containing agnostic data, determine compatibility of the agnostic data files with at least one healthcare resource, characterized in that the devices are arranged to: transmit the agnostic data files to an interoperability platform authorisable by the authorised use to share the agnostic data, and share the agnostic data from the interoperability platform securely via a shared database.

Herein the term ‘secure’ may be construed to include compliance with data protection legislation and comprising the step of validation.

The present interoperability platform comprises data format agnostic means which consolidates and/or synchronizes structured, unstructured, and/or agnostic data,

The system may comprise a computer, portable computing device, and or mobile communicator. The cloud storage system may include the computer, portable computing device, and/or mobile communicator. The cloud storage system may access and control data storage one or more the computer, portable computing device, and/or mobile communicator.

The cloud storage system and/or the digital processor may incorporate a memory and/or processor of one or more of a computer, portable computing device, and/or mobile communicator of one or more individuals, patients and/or users. The cloud storage system and/or the digital processor may comprise one or more persistent data storage devices and/or persistent processor devices which transfer, store, and operate on the structured, unstructured, and/or agnostic data.

According to the method of information management the present interoperability platform may broker the agnostic data securely via a distributed ledger.

The present interoperability platform may be selectively configured for example to:
enable only the authorised user and/or only the patient to authorize sharing the agnostic data and/or patient's medical data from one or more medical providers securely via a shared database; and/or
enable only the authorised user and/or only the patient to authorize sharing the agnostic data and/or patient's medical data from one or more medical authorities securely via a shared database; and/or
transmit information added and/or updated to the agnostic data and/or patient's medical data file by a medical practitioner with authorized access; and/or
share treatment plans and progress updates (the activity and date/time/location) with the patient, individuals, and/or users; and/or
provide the authorised user access to structured data and/or unstructured data in the medical file; and/or
exchange the agnostic data securely between different patient management systems and formats.

The patient management systems and formats may include HL7/MLLP, FHIR, DICOM, and/or SMART. Data exchange is data-format agnostic and data can be securely exchanged between different patient management systems and formats (HL7/MLLP, FHIR, DICOM, SMART etc.) and different storage mechanisms (Cloud based services).

The method may comprise a step in which the present interoperability platform provides pre-processing and post-processing of FHIR server calls to support a plurality of access actions. The present interoperability platform may provide pre-processing and post-processing of FHIR server calls to support a plurality of result filtering actions. The present interoperability platform may be configured to operate at least one consent manager and thereby guarantee access only to authorised users.

The method may include a step wherein a database logs transactions for the purposes of performing audits.

The method may include a step wherein aggregation of data is provided by one or more re-routing microservices which are operative to re-route queries.

The present interoperability platform may be configured, whereby in dependence of a query from an authorised user, the present interoperability platform is converted into multiple requests to a FHIR server. The present interoperability platform is configurable based on a pay-load of queries received. The present interoperability platform may be configured wherein queries are resolved by a combination of synchronized calls to the FHIR server and other supporting microservices.

The present interoperability platform may include a validation step.

The present interoperability platform may include a step of detecting irregular activity or patterns for analysis or to detect malicious attacks.

The present interoperability platform may be configured to enable a patient when uploading a structured document to identify the type of the structured document. The method may be operated by a system comprising a processor which may perform a series of parsing steps which reduce the document into categories.

The present interoperability platform may be configured to enable the authorised user to anonymise specified data and/or redact specified data in the medical file to produce user defined fields of anonymized medical data.

The present interoperability platform may be configured to enable the patient to authorise users to access the information management system to access the structured, unstructured, and/or agnostic data on or associated with the patient. Authorised users include third parties and practitioners (the term practitioner is used here to mean General Practitioners (GP), medical consultants, physiotherapists, physical therapists, pharmacists, dentists, opticians, acute medical facilities, screening and scanning centres and alternative medical practitioners/therapists), hospitals (including emergency departments), pharmaceutical companies and organisations responsible for budgeting and cost management).

The present interoperability platform may control access by the individuals and/or patients and/or users by security processes including blockchain immutability and/or tokenization and/or biometric information unique to the individuals and/or patients and/or users.

The present interoperability platform may be configured to create the profile. During a profile creation phase a user signs-up/registers and may create their profile in the system. The user may obtain a unique user identifier (UUI). Other patient identifiers (i.e., across the various patient management. systems) may be mapped to the UUI and initial identification information is augmented as required, for example with identifiers and/or content compatible with national health and social care quality standards.

The present interoperability platform may comprise a data validation stage and an authentication stage and/or a bi-directional authentication stage prior to authorisation.

The present interoperability platform may be configured to upload ‘supplemental’ information. Supplemental information is used here to refer to non-medical, ad hoc information.

The present interoperability platform may be enabled via an application program interface (API) and a secure, temporary link to specified data which can optionally expire after a predetermined period (as defined by the individual patient and/or the authorised user).

The present interoperability platform may improve the speed to access appropriate treatment, reduce the time a practitioner spends on administrative tasks, increase the time the practitioner has to provide care to their patients, inform practitioners' choice of treatment/care pathway and thus the fastest time to recovery (TTR).

The invention will now be described, by way of example only, with reference to the accompanying figures in which:

### BRIEF DESCRIPTION OF THE FIGURES

FIG. 1 shows a system comparison overview;

FIG. 2 shows the standard FHIR Object graph;

FIG. 3 shows the FHIR Object graph;

FIG. 4 shows the Data Analyzer process;

FIG. 5 shows a sample of a parse-able medical record; and

FIG. 6 shows a sample of the precomputed medical model.

### DETAILED DESCRIPTION OF THE INVENTION

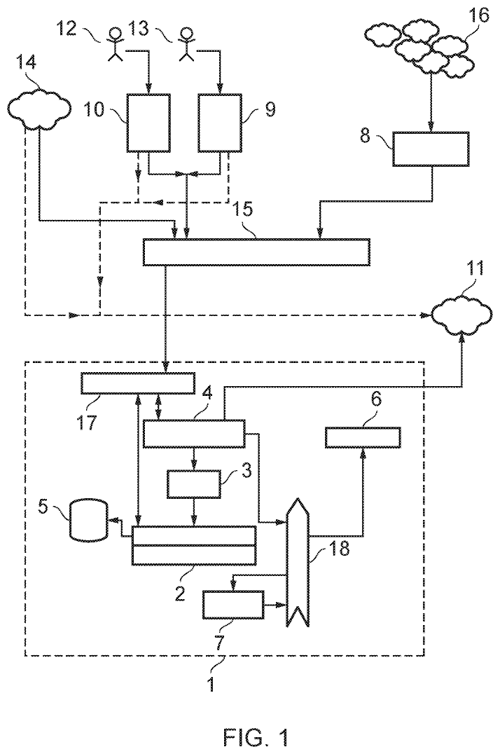

With reference to the drawing, FIG. 1 shows an arrangement of the present healthcare platform-as-a-service interoperability platform, comprising a device, desk top and/or mobile application front end (FIG. 1 (10)), web portal (FIG. 1 (9)) and API (FIG. 1 (4)) for a patient to make and manage appointments, make payments, access their health record, receive prescriptions, receive video content, create entries for previous medical treatment, enter personal information, define preferred practitioners, find practitioners with capacity, complete triage service, receive pre-and post-consult information and receive reminders, manage consents and to grant access to practitioners to view the patient's healthcare information.

At the core of the system (FIG. 1 (1)) is a set of servers (FIG. 1 (2,4,3,6)) and databases (FIG. 1 (5)) with sufficient memory capacity to store the health care data for all member patients. By default, each member patient's data is private. Sharing of data between members (patient, practitioner/organization) is controlled by a consent system (FIG. 1 (3)) that provides granular identity and time-based access to member data. The term ‘member data’ refers to both personal health information (PHI) and non-PHI data. For example: a member can create a consent to share data related to their current medication for a period of 1 week to a specific practitioner. All requests to the system (FIG. 1 (1)) are overseen by this consent system (FIG. 1 (3)).

It is an advantage of the current interoperability platform that individuals providing consent do not necessarily have to be members of the system. Instead, they can access shared data through a password-secured unique URI.

A further advantage of the present interoperability platform is its tracking and auditing data permissions and consents. This not only provides a validated ‘paper trail’ of all activity on the system but can also be used as a source of training data and input data for Al driven validation of permissions and consents.

A Data Integration service (FIG. 1 (8)) connects the core system to information, including from wearable medical devices, medical practice software applications and any other third-party medical data sources that can be integrated, to be normalized to the FHIR schema and stored securely in the system (FIG. 1 (1)). The patient can also submit data to the system (FIG. 1 (1)) to be stored as part of their electronic health record.

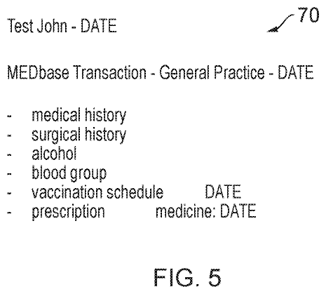

On upload by a patient of a structured document (e.g., a medical record or consultation) the system identifies the type of document and through a series of parsing steps, breaks the document into categories and/or a chronological order. For example, a Data Integration service (FIG. 1 (8)) can accept an uploaded medical record (FIG. 5) and locate the header contexts (FIG. 5 (1,2)), match them against known headers and then take the bodies (FIG. 5 (3,4)) and parse the body given the context of the header. In this example (FIG. 5 (2,3)) the header indicates that the body is a list of vaccines. The body is parsed as a list of vaccines, each vaccine indicated by the ‘vaccine’title in the left-hand column.

In an embodiment a desktop application enables integration of practitioners' software solutions and sharing of consultation notes (including via dictation), triage, pre-and post-consult content, referrals, prescriptions and video content (for example therapy/instructional and/or educational video content), including sharing between the mobile app and the practitioner application.

In an embodiment the system (FIG. 1 (1)) will trigger events upon each change in patient data, analyze them (FIG. 1 (6)) and may indicate or otherwise alert a practitioner and/or patient to consider an action. The information may also be analyzed for treatment efficacy, combined with computed trending information and/or a scoring element to inform a practitioner's decisions.

New data derived from analysis (FIG. 1 (6)) is also stored as part of the patient's electronic health record.

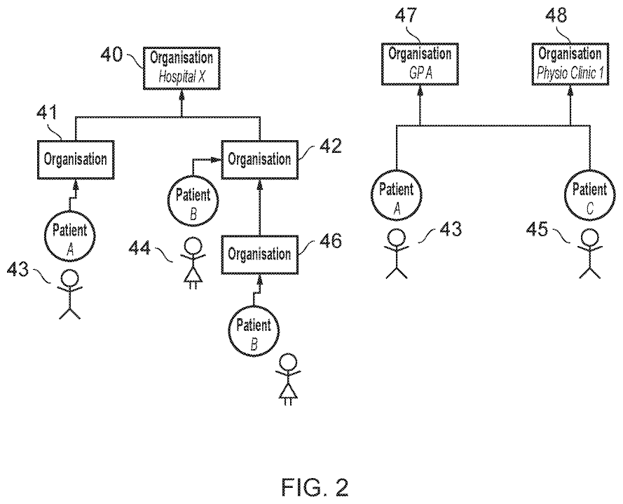

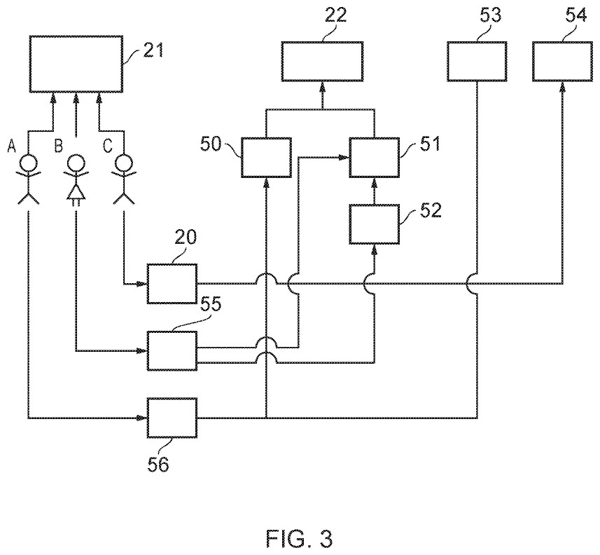

With reference to the drawings, FIG. 2 and FIG. 3, FIG. 2 shows the high level FHIR object graph. The FHIR paradigm is centered around the Organization (for example: hospital, GP practice) with the Patient as a ‘child’ of the Organization. This results in a real-world patient being replicated in each FHIR deployment resulting in sharing of the patient's full medical record. For example, as shown in FIG. 2 Patient A has data in 2 separate FHIR Organizations.

In an embodiment the system (FIG. 1 (1)) the FHIR object graph is organized as shown in FIG. 3. A virtual FHIR organization (21) is created in which all the members are placed. Links to each patient's real world FHIR data with Organizations (22) are established using Consent objects (20). It is an advantage of this graph arrangement that a patient may belong to one Organization and have all their health data under that Organization. It is also an advantage that this graph arrangement is a valid FHIR configuration and permits the virtual Organization (21) to interact with other real-world FHIR organizations (22) using standard FHIR interactions.

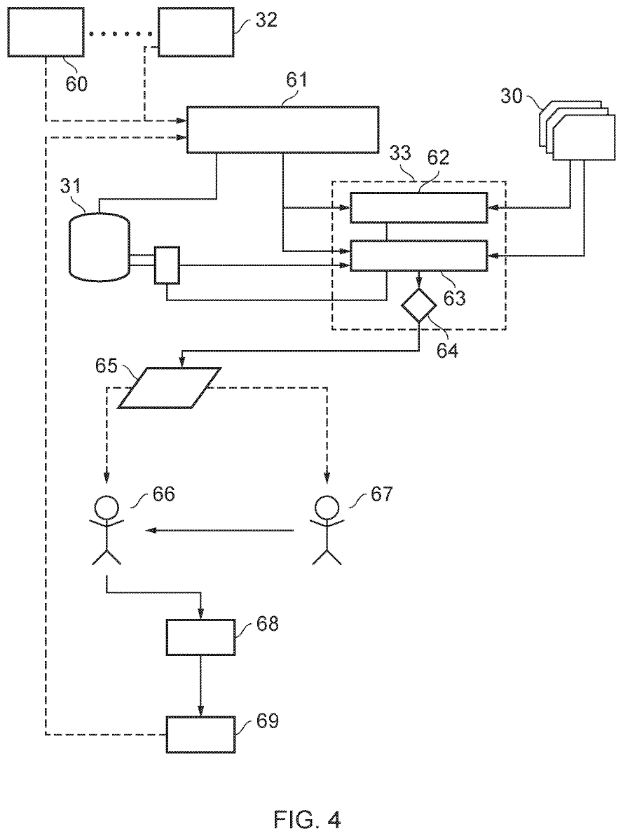

With reference to the drawing, FIG. 4, shows the data analysis process carried out by the Data Analyzer (FIG. 4 (33), FIG. 1 (6)). Every time data is created or updated (FIG. 4 (32)) in the system (FIG. 1(1)) the Data Analyzer (FIG. 4 (33)) is invoked.

Additionally, the present invention incoming data changes or creations are compared with existing patient health records. Initially, the comparison process involves analyzing trends within homogeneous health data. These trends are then captured and stored as new patient health records within the system (refer to FIG. 4 (31)) becoming part of the patient's full medical history and thus able to be shared like other of the patient's health records.

Additionally, the invention includes computing scores by comparing a subset of the patient's current health metrics with a precomputed health model (refer to FIG. 4 (30)). These scores are subsequently stored as new patient health records within the system (refer to FIG. 4 (31)) again becoming part of the patient's full medical history,

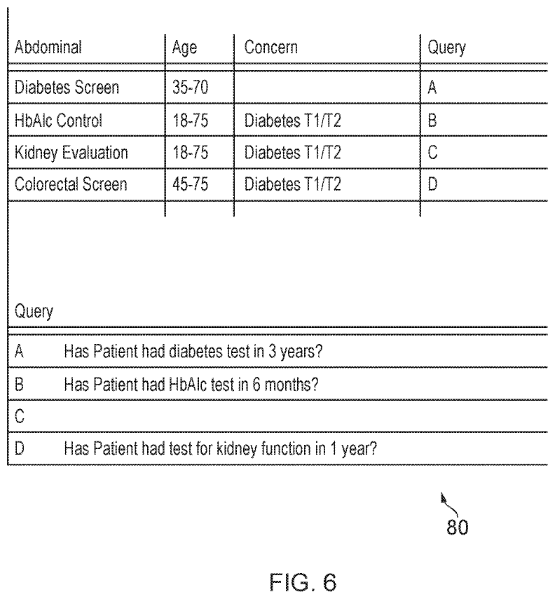

Additionally, advisory treatments/investigations are determined through computational processes. This involves integrating recent updates in health records with the current patient health data, followed by a matrix threshold analysis against the internal medical data model (refer to FIG. 4 (30)). A sample of the internal medical model can be seen in (FIG. 6). Upon meeting or surpassing a specified threshold, an advisory health record, such as a recommendation for an eye scan, is generated, and both the patient and practitioner are promptly notified. Referring to (FIG. 6 (1)) as an example, if a patient is aged 45, male or female, does not have an existing diabetic condition, has an elevated BMI and has no record of diabetic screening in the last 3 years then a recommendation of diabetic screening is recorded. Both the patient and their GP are notified of this recommendation.

In cases where the patient has already completed the recommended treatment/investigation, they have the option to upload the results as per standard procedure.

If they have not completed the recommended treatment/investigation, their practitioner can arrange an appointment. Once attended the data integrations (FIG. 4 (32)) will pick up the results and feed back into the system and the process continues as such.

It is an advantage of the Data Analyzer (FIG. 4 (33)) that a treatment plan and a patient's progress throughout the treatment is tracked, prompts are sent to the patient, and the GP receives confirmation of progress or lack of action/progress.

It is an advantage of the system that it supports the GP, feeding into interpretation of a patient's whole health context and that the GP has the ultimate decision.

The system can operate on a survey, a particular practice management system for the particular patient, patient inputs and/or outputs, practitioner inputs and/or outputs, appointment information, patient form and treatment notes, patient allergy information, consultation information, documents related to the patient or practitioner for or as result of the consultation which may include information about immunization, prescription, procedure, and/or medical history.

In an embodiment data anonymization (FIG. 1 (7)) may be via means akin to ‘Blockchain’ methodologies and identification of an individual/patient only occurs at the application level when the data is accessed by the respective individual/patient or practitioner, thus enabling the holding of whole of life healthcare data across all interactions.

In an embodiment data anonymization enables further valuable uses cases such as population-based analysis of the member cohort for use within medical research, insurance risk assessment modelling, risk group identification and direct messaging.

In an embodiment the present interoperability platform is configured so that in some circumstances an individual query to the present interoperability platform is converted into multiple requests.

In an embodiment the present interoperability platform is configured whereby, based on the payload of the queries received, some queries are resolved by a combination of subqueries to the server and other supporting microservices. This supports the requirement whereby a patient's medical data may be regionally sharded for data protection reasons can be accessed via a single query to the FHIR API.

In an embodiment the present interoperability platform is configured whereby, based on the payload of the queries received, some queries are resolved by a combination of subqueries to the FHIR server and enqueuing of the message within a specific data bus.

### ANNOTATED FEATURES IN FIGURES

1 Servers of system
2 Application Server including application logic
3 Consent Engine
4 FHIR Api
5 Local Database
6 Data Analyzer
7 Data Anonymizer
8 Data Integration Feed
9 Control Web Portal
10 Control Mobile App
11 Identity Provider
12 Patient
13 Practitioner
14 Third party Api user
15 Web application firewall
16 Discrete: wearable, clinic, GP practice, hospital, lab
17 Api manager
18 Service bus
20 Consent Patient C, Observation Z, Physio Clinic 1
21 Organisation—Control
22 Organisation—Hospital X
30 Control Medical Model
31 Patient EHRs
32 Data Integration Feed Xn
33 Data Analyzer
40 Organisation—Hospital X
41 Organisation—sub 1
42 Organisation—sub 2
43 Patient A
44 Patient B
45 Patient C
46 Organisation—sub 3
47 Organisation—GP A
48 Organisation—Physio Clinic 1
50 Organisation—sub 1
51 Organisation—sub 2
52 Organisation—sub 3
53 Organisation—GP A
54 Organisation—Physio Clinic 1
55 Consent Patient B, Observation Z, Physio Clinic 1
56 Consent Patient A, Observation Z, Physio Clinic 1
60 Data Integration Feed X1
61 FHIR
62 Trending/Scoring
63 Thresholding
64 Meets Threshold Query
65 New Medical Observation Suggested
66 Patient
67 Practitioner
68 Facility
69 Data Integration Feed Xn
70 Parse-able medical record
80 Precomputed medical model.

The invention has been described by way of examples only. Therefore, the foregoing is considered as illustrative only of the principles of the invention. Further, since numerous modifications and changes will readily occur to those skilled in the art, it is not desired to limit the invention to the exact construction and operation shown and described, and accordingly, all suitable modifications and equivalents may be resorted to, falling within the scope of the claims.

1. A data format agnostic system for secure individual patient-controlled health information management, comprising devices arranged to:
create a profile unique to each individual patient,
authenticate the identity of the individual patient,
provide at least one authorized user secure access to at least a portion of an existing medical data file of the individual patient,
collate authorized user defined fields of medical data as agnostic data files containing agnostic data,
determine compatibility of the agnostic data files with at least one healthcare resource, characterized in that the devices are arranged to:
transmit the agnostic data files to an interoperability platform authorizable by the authorized use to share the agnostic data, and
share the agnostic data from the interoperability platform securely via a shared database.

2. The data format agnostic system according to claim 1 wherein the devices comprise the interoperability platform.

3. The data format agnostic system according to claim 1 wherein the interoperability platform comprises a data format agnostic means which consolidates and/or synchronizes the agnostic data.

4. The data format agnostic system according to claim 3 wherein the interoperability platform consolidates and/or synchronizes structured and/or unstructured data.

5. The data format agnostic system according to claim 2 wherein the interoperability platform brokers the agnostic data securely via a distributed ledger.

6. The data format agnostic system according to claim 2 wherein the interoperability platform is selectively configured to enable only the authorized user and/or only the individual patient to authorize sharing the agnostic data and/or the medical data file with another authorized user (medical providers, authorities and practitioners) one or more medical authorities securely via a shared database.

7-8. (canceled)

9. The data format agnostic system according to claim 2 wherein the interoperability platform is selectively configured to share treatment plans and progress updates including activity, date, time, and, or location with the individual patient, another person, and/or another user of the data format agnostic system.

10. The data format agnostic system according to claim 4 wherein the interoperability platform is selectively configured to provide the authorized user access to structured data and/or unstructured data in the medical file.

11. The data format agnostic system according to claim 2 wherein the interoperability platform is selectively configured to exchange the agnostic data securely between different patient management systems and formats.

12. (canceled)

13. The data format agnostic system according to claim 2 wherein the interoperability platform is selectively configured to process and resolve queries from the authorized user using consent objects by a combination of synchronized calls to a FHIR server and other supporting microservices.

14. (canceled)

15. The data format agnostic system according to claim 2 wherein the interoperability platform is selectively configured wherein a virtual Fast Health Interoperability Resources (FHIR) organization is created in which all the individual patients and authorized users who members of the system are placed.

16. The data format agnostic system according to claim 15 wherein the interoperability platform is selectively configured wherein queries resolved by a combination of subqueries to the FHIR server and enqueuing of the message within a specific data bus.

17. The data format agnostic system according to claim 2 wherein the interoperability platform is configured to execute a validation step.

18. The data format agnostic system according to claim 2 wherein the interoperability platform is configured to execute a step of detecting irregular activity or patterns for analysis or to detect malicious attacks.

19. (canceled)

20. The data format agnostic system according to claim 2 wherein the interoperability platform is configured to anonymize specified data and/or redact specified data in the medical data file to produce user defined fields of anonymized medical data.

21-25. (canceled)

26. The data format agnostic system according to claim 2 wherein the interoperability platform is operable via an application program interface (API) and a secure, temporary link to specified data which expires after a predetermined period.

27. (canceled)

28. The data format agnostic system according to any claim 1 comprising at least one server and at least one database with memory to store the profile, medical data file, and agnostic data files

29. The data format agnostic method for secure individual patient-controlled health information management implemented the data format agnostic system according to claim 1.

30. The data format agnostic system according to claim 2 wherein the interoperability platform is configured to authorize access to at least a portion of an existing medical data file.
